SEQUENCE LISTING (21 sequences)

SEQ ID NO: 1 - protein, 63 aa, Homo sapiens
GVTLFVALYDYEARTEDDLSFHKGEKFQILNSSEGDWWEARSLTTGETGYIPSNYVAPVDSIQ

SEQ ID NO: 2 - protein, 46 aa, Homo sapiens
YSSPGSPGTPGSRSRTPSLPTPPTREPKKVAVVRTPPKSPSSAKSR

SEQ ID NO: 3 - DNA, 189 bp, Homo sapiens
ggagtgacactctttgtggccctttatgactatgaagcacggacagaagatgacctgagttttcacaaaggagaaaaatttcaaatattgaacagctcggaaggagattggtgggaagcccgctccttgacaactggagagacaggttacattcccagcaattatgtggctccagttgactctatccag

SEQ ID NO: 4 - DNA, 138 bp, Homo sapiens
tacagcagccccggctccccaggcactcccggcagccgctcccgcaccccgtcccttccaaccccacccacccgggagcccaagaaggtggcagtggtccgtactccacccaagtcgccgtcttccgccaagagccgc

SEQ ID NO: 5 - protein, 158 aa, Aequorea victoria
MVSKGEELFTGVVPILVELDGDVNGHKFSVSGEGEGDATYGKLTLKFICTTGKLPVPWPTLVTTFTYGVQCFARYPDHMKQHDFFKSAMPEGYVQERTIFFKDDGNYKTRAEVKFEGDTLVNRIELKGIDFKEDGNILGHKLEYNYNSHKVYITADKQ

SEQ ID NO: 6 - DNA, 474 bp, Aequorea victoria
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcag

SEQ ID NO: 7 - protein, 81 aa, Aequorea victoria
KNGIKVNFKTRHNIEDGSVQLADHYQQNTPIGDGPVLLPDNHYLSTQSALSKDPNEKRDHMVLLEFVTAAGITLGMDELYK

SEQ ID NO: 8 - DNA, 246 bp, Aequorea victoria
aagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 9 - DNA, 32 bp, Artificial Sequence
  Artificial sequence derived from Aequorea victoria
gggaattcaccatggtgagcaagggcgaggag

SEQ ID NO: 10 - DNA, 42 bp, Artificial Sequence
  Artificial sequence derived from Aequorea victoria
ggtctagagccggagccgccctgcttgtcggcggtgatatag

SEQ ID NO: 11 - DNA, 501 bp, Artificial Sequence
  Artificial sequence derived from Aequorea victoria
gaattcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcacctacggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaaggtctatatcaccgccgacaagcagggcggctccggctctaga

SEQ ID NO: 12 - protein, 164 aa, Artificial Sequence
  Artificial sequence derived from Aequorea victoria
MVSKGEELFTGVVPILVELDGDVNGHKFSVSGEGEGDATYGKLTLKFICTTGKLPVPWPTLVTTFTYGVQCFARYPDHMKQHDFFKSAMPEGYVQERTIFFKDDGNYKTRAEVKFEGDTLVNRIELKGIDFKEDGNILGHKLEYNYNSHKVYITADKQGGSGSR

SEQ ID NO: 13 - DNA, 35 bp, Artificial Sequence
  Artificial sequence derived from Aequorea victoria
gggaattcaccatgaagaacggcatcaaggtgaac

SEQ ID NO: 14 - DNA, 42 bp, Artificial Sequence
  Artificial sequence derived from Aequorea victoria
ggtctagagccggagccgcccttgtacagctcgtccatgccg

SEQ ID NO: 15 - DNA, 273 bp, Artificial Sequence
  Artificial sequence derived from Aequorea victoria
gaattcaccatgaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggcggctccggctctaga

SEQ ID NO: 16 - protein, 88 aa, Artificial Sequence
  Artificial sequence derived from Aequorea victoria
MKNGIKVNFKTRHNIEDGSVQLADHYQQNTPIGDGPVLLPDNHYLSTQSALSKDPNEKRDHMVLLEFVTAAGITLGMDELYKGGSGSR

SEQ ID NO: 17 - DNA, 44 bp, Artificial Sequence
  Artificial sequence derived from Aequorea victoria
ggtctagaggcggctccggcaagaacggcatcaaggtgaacttc

SEQ ID NO: 18 - DNA, 31 bp, Artificial Sequence
  Artificial sequence derived from Aequorea victoria
ggggatccttacttgtacagctcgtccatgc

SEQ ID NO: 19 - DNA, 270 bp, Artificial Sequence
  Artificial sequence derived from Aequorea victoria
tctagaggcggctccggcaagaacggcatcaaggtgaacttcaagacccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaggatcc

SEQ ID NO: 20 - protein, 87 aa, Artificial Sequence
  Artificial sequence derived from Aequorea victoria
SRGGSGKNGIKVNFKTRHNIEDGSVQLADHYQQNTPIGDGPVLLPDNHYLSTQSALSKDPNEKRDHMVLLEFVTAAGITLGMDELYK

SEQ ID NO: 21 - protein, 19 aa, Homo sapiens
YGRKKRRQRRRTPPKSPSS

(19) United States
(12) Patent Application Publication
(10) Pub. No.: US 2015/0017657 A1
(43) Pub. Date: Jan. 15, 2015

(54) ASSAY AND METHOD FOR IDENTIFYING COMPOUNDS THAT INHIBIT EXCITOTOXIC SIGNALS

(71) Applicant: THE UNIVERSITY OF SYDNEY, New South Wales (AU)

(72) Inventors: Lars Matthias Ittner, Camperdown (AU); Juergen Goetz, Brookfield (AU)

(73) Assignees: JUERGEN GOETZ, BROOKFIELD, QLD (AU); LARS MATTHAIS ITTNER, CAMPERDOWN, NSW (AU)

(21) Appl. No.: 14/365,797

(22) PCT Filed: Dec. 14, 2012
(86) PCT No.: PCT/AU2012/001540

§ 371 Date: Jun. 16, 2014

(30) Foreign Application Priority Data
Dec. 16, 2011 (AU) 2011905260

(51) Int. Cl. G01N 33/50; C07K 14/47

(52) U.S. Cl. CPC G01N 33/5032; C07K 14/4711; G01N 2500/02; G01N 2500/10; G01N 2333/47
USPC 435/7.8; 536/23.5; 435/320.1

(57) ABSTRACT

The invention relates to an assay for identifying compounds for the treatment of various diseases including those associated with excitotoxicity. The invention also relates to cell lines and constructs for use in an assay of the invention. The invention also relates to methods for determining whether a compound reduces excitotoxic signalling in a cell and whether a compound that inhibits binding of Tau to Fyn is likely to selectively reduce excitotoxic cell signalling. The invention also relates to a Tau protein adapted to form a detectable signal when the Tau protein is bound to a Fyn protein. The invention also relates to a Fyn protein adapted to form a detectable signal when the Fyn protein is bound to a Tau protein.

### FIELD OF THE INVENTION

The invention relates to an assay for identifying compounds for the treatment of various diseases including those associated with excitotoxicity. The invention also relates to cell lines and constructs for use in an assay of the invention.

### BACKGROUND OF THE INVENTION

Reference to any prior art in the specification is not, and should not be taken as, an acknowledgment or any form of suggestion that this prior art forms part of the common general knowledge in Australia or any other jurisdiction or that this prior art could reasonably be expected to be ascertained, understood and regarded as relevant by a person skilled in the art.

Nerve cells and tissues may be damaged and killed by glutamate and similar substances when receptors for excitatory transmitters, examples being the NMDA receptor and AMPA receptor are over-activated. According to the process, NMDA, kainic acid and other molecules that bind to these types of receptors, as well as pathologically high levels of glutamate facilitate the ingress of calcium ions into a cell, leading to activation of enzymes such as phospholipases, endonucleases, proteases and concomitant damage to cytoskeleton, membrane and DNA.

The pathology is known as “excitotoxicity” and it is believed to be involved in many diseases, conditions and syndromes of the nervous system including spinal cord injury, stroke, traumatic brain injury, MS, Alzheimer's disease, ALS, Parkinson's disease, Huntington's disease, and other neurodegenerative diseases.

At a molecular level, the phosphorylation of residues on NMDA receptors by the tyrosine kinase Fyn is understood to be an important event in the transmission of certain excitotoxic signals. This event is understood to be important for the interaction of proteins such as PSD-93, PSD-95 and nNOS with NMDA receptor.

One approach to blocking the transmission of an excitotoxic signal has been to introduce a peptide into a cell, the peptide having a sequence that is more or less identical to a region of a NMDA receptor (a NR2B receptor) containing a residue for phosphorylation by Fyn such as a tyrosine residue. It is believed that saturation of a compartment of a neuron at a post synaptic cleft where NR2B receptor is located blocks the phosphorylation of a subject tyrosine on the NR2B receptor by Fyn, probably by creating competition for phosphorylation by Fyn that is skewed in favour of phosphorylation of the peptide. It is not clear whether this approach would be useful for blocking transmission of excitotoxic signals in the treatment of a condition or disease because without adequate delivery and saturation at a majority of post synaptic clefts, it would be possible for Fyn to phosphorylate a NMDA receptor tyrosine residue leading to interaction with proteins such as PSD-93, PSD-95 and nNOS and generation of the signal.

Another approach is to introduce a peptide that has a sequence for binding to molecular domains on either side of the PSD-95/NMDA receptor interaction complex. Again this approach does not stop Fyn from phosphorylating NR2B so signal transmission remains possible without adequate delivery and saturation at a majority of post synaptic clefts.

WO 2009/143556 describes that the excitotoxic signal transmission can be inhibited by sequestering Fyn in a nerve cell soma so as to prevent localisation of Fyn at the post synaptic cleft. This sequestration can be achieved by the Tau projection domain.

There is a need for assays and related methods for identifying compounds that are likely to block excitotoxic signals.

There is also a need for assays and related methods for identifying compounds that are likely to selectively inhibit excitotoxic signalling, i.e. compounds that block excitotoxic signalling and that have minimal effect on normal constitutive signalling.

### SUMMARY OF THE INVENTION

The invention seeks to address at least one of the above identified needs and in one embodiment provides a method for determining whether a compound reduces excitotoxic signalling including the steps of:
providing a compound for which a capacity to reduce excitotoxic signalling is to be determined;
providing a Tau protein and a Fyn protein, wherein the Tau protein includes a domain for binding the Fyn protein, and wherein the Tau and/or Fyn proteins are adapted to form a detectable signal when the Tau protein is bound to the Fyn protein;
contacting the Tau protein and the Fyn protein with the compound in conditions for permitting the compound to bind to either or both of the Tau protein and Fyn protein, thereby inhibiting the binding of the Tau protein to the Fyn protein when the compound is bound to either or both of Tau protein and Fyn protein;
determining whether a detectable signal is formed from binding of Tau protein to Fyn protein;
   wherein an absence of a detectable signal indicates that the compound inhibits the binding of the Tau protein to the Fyn protein,
   thereby determining whether the compound reduces excitotoxic signalling.

In another embodiment there is provided a nucleic acid encoding a Tau protein that is adapted to form a detectable signal when the Tau protein is bound to Fyn protein.

In another embodiment there is provided a nucleic acid encoding a Fyn protein that is adapted to form a detectable signal when the Fyn protein is bound to Tau protein.

In another embodiment there is provided a vector or construct including a nucleic acid described above.

In another embodiment there is provided a cell including a nucleic acid, vector or construct described above.

In further embodiments there is provided a method for determining whether a compound that inhibits binding of Tau to Fyn is likely to selectively reduce excitotoxic signalling comprising the steps of:
providing an inhibitor in the form of a compound that binds to either or both of Tau and Fyn, thereby inhibiting the binding of Tau to Fyn when the compound is bound to either or both of Tau and Fyn;
providing a Tau protein and a Fyn protein, wherein the Tau protein includes a domain for binding to the Fyn protein, and wherein the Tau and/or Fyn proteins are adapted to form a detectable signal when the Tau protein is bound to the Fyn protein;
utilizing the inhibitor to inhibit binding of the Tau protein to the Fyn protein, thereby inhibiting generation of a detectable signal that is formed from binding of the Tau protein to the Fyn protein;
providing conditions for permitting formation of the detectable signal; and
determining whether a detectable signal is formed,
   wherein formation of the detectable signal determines that the compound is likely to selectively reduce excitotoxic cell signalling.

In another embodiment there is provided a kit including:
a nucleic acid, vector or construct, or cell as described above;
written instructions for use in a method described above.

### BRIEF DESCRIPTION OF THE DRAWINGS

FIG. 1: (a) Amino acids 83-145 of human Fyn (SEQ ID NO: 1); and (b) Amino acids 197-242 of human Tau (SEQ ID NO: 2).

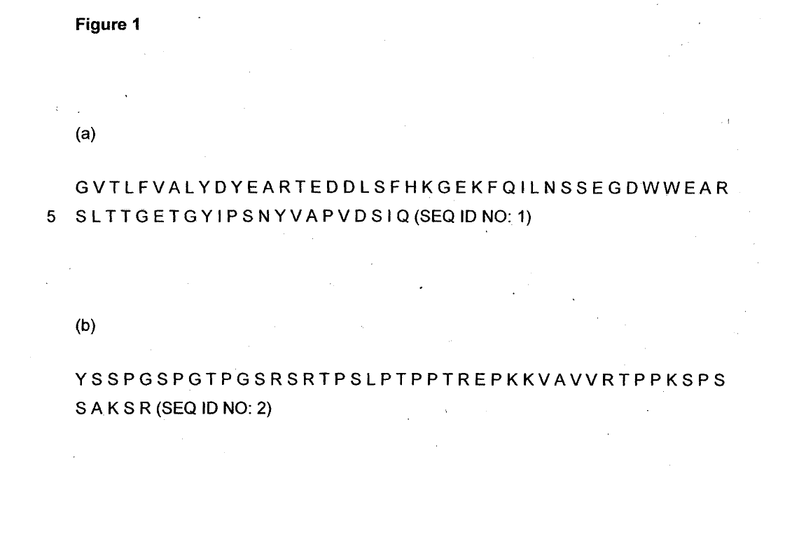

FIG. 2: (a) Nucleotide sequence encoding amino acids 83-145 of human Fyn (SEQ ID NO: 3); (b) nucleotide sequence encoding amino acids 197-242 of human Tau (SEQ ID NO: 4).

FIG. 3: (a) Amino acids 1-158 of GFP which is the amino acid sequence of N-GFP (SEQ ID NO: 5), (b) nucleotide sequence of N-GFP (SEQ ID NO: 6).

FIG. 4: (a) Amino acids 159 to 239 of GFP which is the amino acid sequence of C-GFP (SEQ ID NO: 7), (b) nucleotide sequence of C-GFP (SEQ ID NO: 8).

FIG. 5: Fluorescence microscopy image of COS-7 cells transfected with N-GFP-Fyn-pLVX and C-GFP-Tau-pLVX (a), or left panel, in the absence of Tat-7PXXP and (b), or right panel, in the presence of Tat-7PXXP.

FIG. 6: Schematic of an embodiment of an assay of the invention.

### DETAILED DESCRIPTION OF THE EMBODIMENTS

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described.

It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

As used herein, except where the context requires otherwise, the term “comprise” and variations of the term, such as “comprising”, “comprises” and “comprised”, are not intended to exclude further additives, components, integers or steps.

Excitotoxic signalling is known to be associated with the development and maintenance of a range of pathologies including epilepsy, hypoxia, traumatic injury to the CNS, stroke, Alzheimer's disease and Parkinson's disease. While there are many chemical libraries that might contain compounds for blocking excitotoxicity, at the time of the invention there were no high through put assays that could be used to identify those compounds that are most likely to inhibit excitotoxicity, and from which one could identify candidates for pre-clinical trials in animal models.

The inventors have developed a method for determining whether a compound is likely to reduce excitotoxic signalling. The method is based on the concept that if it is possible to block binding of Tau with Fyn (or Fyn with Tau), it should then be possible to stop Tau from delivering Fyn to the post synaptic membrane where, if so located, Fyn would be able to phosphorylate NMDA receptors leading to excitotoxic signal transduction. Accordingly, the method utilizes the affinity of Tau for Fyn (or Fyn for Tau) to form an assay system whereby when Tau is bound to Fyn (or Fyn is bound to Tau) a detectable signal is formed. The signal is not formed when Tau is not bound to Fyn (or Fyn is not bound to Tau), an outcome that is likely where a compound is bound to either Fyn or Tau in such an arrangement that Tau is then precluded from binding to Fyn (or Fyn is then precluded from binding to Tau). Compounds that prevent the binding of Tau to Fyn (and which hence prevent signal generation) are identified as being likely to reduce excitotoxic signalling because they are likely to prevent location of Fyn to a post synaptic membrane by Tau.

Therefore, in one embodiment there is provided a method for determining whether a compound reduces excitotoxic signalling. Generally the method determines the likelihood of a compound being able to reduce excitotoxic signalling, again based on the understanding that if the compound binds to Tau or Fyn in such a way as Tau is then precluded from binding to Fyn, then Fyn mediated phosphorylation arising from Tau mediated translocation of Fyn to a post synaptic membrane should be precluded. It is in this context that the method is particularly useful for determining likelihood of a compound, especially with regard to other compounds in a chemical library, of reducing excitotoxicity. Having identified compounds that are likely to reduce excitotoxicity, in the form of compounds that block engagement of Tau with Fyn with the method of invention, the relevant compounds identified with the method may then be screened in cell models in which excitotoxic signal transduction can be measured, or in animal models in which signal transduction can be measured, or in which the modification of a pathology associated with signal transduction, such as memory deficit or retention can be measured.

The first step of the method generally involves providing a compound for which a capacity to reduce excitotoxic signalling, or for which likelihood of reducing excitotoxic signal transduction is to be determined. Generally the compound will be provided in the form of a chemical library or fraction thereof. The compound may be one which has already found therapeutic application. Typically the compound is provided in serial dilutions for use in the method of invention, thereby providing for a standard curve against which efficacy for blocking of Tau/Fyn aggregation can be determined against controls. In certain embodiments of the invention the test compound is a small molecule, peptide or a peptidomimetic. A ‘peptidomimetic’ is a synthetic chemical compound that has substantially the same structure and/or functional characteristics of a peptide of the invention, the latter being described further herein. Typically, a peptidomimetic has the same or similar structure as a peptide of the invention. A peptidomimetic generally contains at least one residue that is not naturally synthesised. Non-natural components of peptidomimetic compounds may be according to one or more of: a) residue linkage groups other than the natural amide bond (′peptide bond′) linkages; b) non-natural residues in place of naturally occurring amino acid residues; or c) residues which induce secondary structural mimicry, i.e., to induce or stabilize a secondary structure, e.g., a beta turn, gamma turn, beta sheet, alpha helix conformation, and the like.

As used herein, the term “contacting” refers to the bringing together or combining of molecules such that they are within a distance for allowing of intermolecular interactions such as the non-covalent interaction between a two peptides or one protein and a compound. In some embodiments, contacting occurs in solution phase in which the combined or contacted molecules are dissolved in a common solvent and are allowed to freely associate. In some embodiments, the contacting can occur within a cell or in a cell-free environment. In some embodiments, the cell-free environment is the lysate produce from a cell. In some embodiments, a cell lysate may be a whole-cell lysate, nuclear lysate, cytoplasm lysate, and combinations thereof. In some embodiments, the cell-free lysate is only lysate obtained from a nuclear extraction and isolation wherein the nuclei of a cell population are removed from the cells and then lysed. In some embodiments, the nuclei are not lysed, but are still considered to be a cell-free environment. The interacting molecules can also be mixed such as through vortexing, shaking, and the like.

The next step generally involves the provision of a Tau protein and a Fyn protein. Typically the Tau and Fyn proteins are based on human sequences of these proteins, although in some embodiments, the sequences may be mammalian sequences, and particularly in circumstances where the proteins are expressed in non human cell lines.

The human Tau protein can occur in the brain in six alternatively spliced isoforms. The longest human Tau isoform, htau40 (441 aa) (NCBI sequence reference NP_{—}005901), comprises an amino-terminal projection domain (PD; also known as Tau projection domain or projection domain of Tau), followed by a microtubule binding domain (MTB) with four repeats and a carboxy-terminal tail. The amino-terminal projection domain of Tau protrudes from the microtubule surface when the Tau protein is bound to microtubules.

htau40 can also be referred to as 2N4R as it contains 2 amino-terminal inserts (2N) and 4 microtubule-binding repeats (4R). The two amino-terminal inserts are encoded by two alternatively spliced exons, E2 and E3, and encode 29 amino acids each. The various isoforms of the Tau protein arise from alternative splicing of exon 2, 3 and 10. The isoforms differ in either 0, 1 or 2 inserts of the 29 amino acid amino-terminal part and three or four microtubule-binding repeats. The isoforms of human Tau are summarised below:

The 0N3R isoform is 352 amino acids in length (NCBI sequence reference NP_{—}058525.1), with the amino-terminal projection domain being 197 amino acids.

The 0N4R isoform is 383 amino acids in length (NCBI sequence reference NP 058518.1), with the amino-terminal projection domain being 197 amino acids.

The 1N3R isoform is 383 amino acids in length, with the amino-terminal projection domain being 226 amino acids.

The 1N4R isoform is 412 amino acids in length, with the amino-terminal projection domain being 226 amino acids.

The 2N3R isoform is 410 amino acids in length, with the amino-terminal projection domain being 255 amino acids.

The 2N4R isoform is 441 amino acids in length, with the amino-terminal projection domain being 255 amino acids.

The amino acid sequence of human Tau isoforms can be found in publicly available databases, for example those supported by NCBI (National Center for Biotechnology Information), including GenBank®.

The human Fyn protein can occur in three alternatively spliced isoforms. The longest human Fyn isoform, isoform a (537 aa) (NCBI sequence reference NP_{—}002028.1), contains the following domains: SH3 (aa 87-140), SH2 (aa 145-245) and PTKc-Src-like (aa 264-523).

The human Fyn isoform b (534 aa) (NCBI sequence reference NP_{—}694592.1) differs in the 5′ UTR, and lacks an alternate in-frame exon but includes a different in-frame exon in the central coding region, compared to variant a. The encoded isoform b is shorter than isoform a. The domains are the same.

The human Fyn isoform c (482 aa) (NCBI sequence reference NP_{—}694593.1) differs in the 5′ UTR, and lacks an alternate in-frame exon in the central coding region, compared to variant a, resulting in an isoform c that is shorter than isoform a. The domains are the same.

It will be recognised that sequences that have some homology but not complete identify with any one of the above Tau and Fyn sequences could be used in place of any one of the above Tau or Fyn sequences, provided that the relevant sequences at least have the sequence specificity necessary to provide for the interaction between Tau and Fyn that results in Tau-mediated translocation of Fyn to post synaptic membranes. Generally the regions of the Tau projection domain have no less than 90% homology to a reference sequence. Generally the regions of the Fyn SH3 domain have no less than 90% homology to a reference sequence. Other parts of Tau and Fyn may have lesser homology with a Tau or Fyn reference sequence.

In one embodiment the Fyn domain includes, consists essentially of or consists of an amino acid sequence that mediates non-covalent association with the Tau. Preferably, the Fyn domain does not include the full length Fyn protein, however the Fyn domain includes all amino acids of the full length Fyn protein that mediate the native association with Tau. Typically, the Fyn domain includes, consists essentially of or consists of the SH3 domain. Preferably, the amino acid sequence of the Fyn domain includes, consists essentially of or consists of any amino acid sequence that mediates binding to Tau with a similar affinity to amino acids 88 to 145 of human Fyn (SEQ ID NO: 1). Those amino acids of Fyn which do not mediate the interaction with Tau may be substitutable. Even more preferably, the Fyn domain includes, consists essentially of or consists of amino acids 83 to 145 of human Fyn (SEQ ID NO: 1). The Fyn domain may include, consist essentially of or consist of an amino acid sequence that is 60, 70, 80, 90, 91, 92, 93, 94, 95, 96, 97, 98, 99 or 100% identical to the sequence shown in SEQ ID NO: 1.

In one embodiment, the Fyn domain is an isolated, recombinant or synthetic peptide or peptidomimetic.

In one embodiment the Tau domain includes, consists essentially of or consists of an amino acid sequence that mediates non-covalent association with the kinase Fyn. Preferably, the Tau domain does not include the full length Tau protein, however the Tau domain includes all amino acids of the full length Tau protein that mediate the native association with Fyn. Typically, the Tau domain includes, consists essentially of or consists of the amino terminal projection domain. Preferably, the Tau domain includes the seventh PXXP motif of the Tau projection domain. Preferably, the Tau domain includes any amino acid sequence that mediates binding to Fyn with a similar affinity as the amino acid sequence 197 to 242 of human Tau (SEQ ID NO: 2). Those amino acids of Tau which do not mediate the interaction with Fyn may be substitutable, of the seven PXXP motifs the seventh is essential for an interaction of Tau with Fyn. Even more preferably, the Tau domain includes, consists essentially of or consists of the amino acid sequence of 197 to 242 of human Tau (SEQ ID NO: 2). The Tau domain may include, consisting essentially of or consist of an amino acid sequence that is 60, 70, 80, 90, 91, 92, 93, 94, 95, 96, 97, 98, 99 or 100% identical to the sequence shown in SEQ ID NO: 2.

By utilizing only the minimal region of Tau that mediates binding to Fyn and the minimal region of Fyn binding to Tau, it is more likely that a method of the invention will identify inhibitors that directly interfere with the non-covalent protein-protein interaction of Tau and Fyn. However, if more of the Tau protein than just the minimal region that mediates binding to Fyn and if more of the Fyn protein than just the minimal region that mediates the binding to Tau is used in the method of the invention then there is an increased likelihood that an inhibitor which has an allosteric mechanism will be identified. In this context allosteric means disruption of the interaction of Tau and Fyn without directly competing with Tau binding to Fyn or Fyn binding to Tau.

In one embodiment, the Tau domain is an isolated, recombinant or synthetic peptide or peptidomimetic.

“Percent (%) amino acid sequence identity” or “percent (%) identical” with respect to a peptide or polypeptide sequence, i.e. a peptide of the invention defined herein, is defined as the percentage of amino acid residues in a candidate sequence that are identical with the amino acid residues in the specific peptide or polypeptide sequence, i.e. a peptide of the invention, after aligning the sequences and introducing gaps, if necessary, to achieve the maximum percent sequence identity, and not considering any conservative substitutions as part of the sequence identity.

Those skilled in the art can determine appropriate parameters for measuring alignment, including any algorithms (non-limiting examples described below) needed to achieve maximal alignment over the full-length of the sequences being compared. When amino acid sequences are aligned, the percent amino acid sequence identity of a given amino acid sequence A to, with, or against a given amino acid sequence B (which can alternatively be phrased as a given amino acid sequence A that has or comprises a certain percent amino acid sequence identity to, with, or against a given amino acid sequence B) can be calculated as: percent amino acid sequence identity=X/Y100, where X is the number of amino acid residues scored as identical matches by the sequence alignment program's or algorithm's alignment of A and B and Y is the total number of amino acid residues in B. If the length of amino acid sequence A is not equal to the length of amino acid sequence B, the percent amino acid sequence identity of A to B will not equal the percent amino acid sequence identity of B to A.

In calculating percent identity, typically exact matches are counted. The determination of percent identity between two sequences can be accomplished using a mathematical algorithm. A nonlimiting example of a mathematical algorithm utilized for the comparison of two sequences is the algorithm of Karlin and Altschul (1990) Proc. Natl. Acad. Sci. USA 87:2264, modified as in Karlin and Altschul (1993) Proc. Natl. Acad. Sci. USA 90:5873-5877. Such an algorithm is incorporated into the BLASTN and BLASTX programs of Altschul et al. (1990) J. Mol. Biol. 215:403. To obtain gapped alignments for comparison purposes, Gapped BLAST (in BLAST 2.0) can be utilized as described in Altschul et al. (1997) Nucleic Acids Res. 25:3389. Alternatively, PSI-Blast can be used to perform an iterated search that detects distant relationships between molecules. See Altschul et al. (1997) supra. When utilizing BLAST, Gapped BLAST, and PSI-Blast programs, the default parameters of the respective programs (e.g., BLASTX and BLASTN) can be used. Alignment may also be performed manually by inspection. Another non-limiting example of a mathematical algorithm utilized for the comparison of sequences is the ClustalW algorithm (Higgins et al. (1994) Nucleic Acids Res. 22:4673-4680). ClustalW compares sequences and aligns the entirety of the amino acid or DNA sequence, and thus can provide data about the sequence conservation of the entire amino acid sequence. The ClustalW algorithm is used in several commercially available DNA/amino acid analysis software packages, such as the ALIGNX module of the Vector NTI Program Suite (Invitrogen Corporation, Carlsbad, Calif.). After alignment of amino acid sequences with ClustalW, the percent amino acid identity can be assessed. A non-limiting example of a software program useful for analysis of ClustalW alignments is GENEDOC™. GENEDOC™ allows assessment of amino acid (or DNA) similarity and identity between multiple proteins. Another non-limiting example of a mathematical algorithm utilized for the comparison of sequences is the algorithm of Myers and Miller (1988) CABIOS 4:11-17. Such an algorithm is incorporated into the ALIGN program (version 2.0), which is part of the GCG Wisconsin Genetics Software Package, Version 10 (available from Accelrys, Inc., 9685 Scranton Rd., San Diego, Calif., USA). When utilizing the ALIGN program for comparing amino acid sequences, a PAM 120 weight residue table, a gap length penalty of 12, and a gap penalty of 4 can be used.

In accordance with the second step of the method, the Tau and/or Fyn proteins are adapted to form a detectable signal when the Tau protein is bound to the Fyn protein. A “Detectable signal” as used herein refers to any observable effect including enzymatic activity, bioluminescence, chemiluminescence, fluorescence or absorbance. The detectable signal may arise from a split reporter system. Examples of split reporter systems include ubiquitin (Johnsson, N.; Varshaysky, A. Proc Natl Acad Sci USA 1994, 91, 10340-4.), beta-galactosidase (Rossi, F.; Charlton, C. A.; Blau, H. M. Proc Natl Acad Sci USA 1997, 94, 8405-10.), dihydrofolate reductase (Pelletier, J. N.; Campbell-Valois, F. X.; Michnick, S. W. Proc Natl Acad Sci USA 1998, 95, 12141-6.), beta-lactamase (Galameau, A.; Primeau, M.; Trudeau, L. E.; Michnick, S. W. Nat Biotechnol 2002, 20, 619-22.), GFP (Ghosh, I.; Hamilton, A. D.; Regan, L. J. Am. Chem. Soc. 2000, 122, 5658-5659), GFP-variants (MacDonald, M. L.; Lamerdin, J.; Owens, S.; Keon, B. H.; Bilter, G. K.; Shang, Z.; Huang, Z.; Yu, H.; Dias, J.; Minami, T.; Michnick, S. W.; Westwick, J. K. Nat Chem Biol 2006, 2, 329-337. Hu, C. D.; Kerppola, 1. K. Nat Biotechnol, 2003, 21, 539-45.), firefly luciferase (Paulmurugan, R; Umezawa, Y.; Gambhir, S. S. Proc Natl Acad Sci USA 2002, 99, 15608-13) and Gaussia luciferase (Remy, I.; Michnick, S. W. Nat Methods 2006, 3, 977-9).

One example of an adaptation of Tau and Fyn that provides for formation of a detectable signal when Tau is bound to Fyn is described in the examples herein. According to the example, fragments of green fluorescent protein (GFP), each having no capacity for fluorescence alone, but with capacity for fluorescence when combined, are linked to one or other of Tau and Fyn, so that when Tau is bound to Fyn (or Fyn to Tau), the fragments of GFP are brought together, ostensibly re-assembling the GFP thereby generating a fluorescent signal.

It is a particularly surprising finding of the invention that the affinity of Tau for Fyn is sufficient to provide for the necessary molecular interaction between fragments of GFP required for reassembly of a functional GFP. In particular, it is known that the Tau/Fyn interaction is very low affinity, whereas all other examples of a re-assembly reporter assay have been at a much higher affinity. 3R Tau (Kd: 0.326 microM) has higher affinity than 4R Tau (Kd: 7.77 microM), AT8 pseudophosphorylation on 3R Tau backbone reduces affinities up to 95-fold; opposite situation with 4R backbone (Bhaskar et al., JBC 280 (2005) 35119).

Another surprising finding is that the steric, constraints imposed by the Tau/Fyn interaction on GFP fragments linked to these proteins do not hinder the re-assembly of the GFP. This was unanticipated at the time of the invention.

Also, it was also surprising that fusion of a portion of GFP to a domain of Tau and Fyn did not alter the structure of the Tau and Fyn domains such that GFP fused Tau and Fyn could still interact. In addition, the recreation of a detectable signal by a re-assembled or re-constituted GFP requires correctly folded Tau and Fyn domain and GFP. It was unexpected that the folding of all domains was unaffected by their fusion. It was also unexpected that the expression level of the Tau and Fyn domain fusions was of a sufficient level to allow visualisation of the re-assembled or re-constituted GFP in a cell. Finally, it was surprising that the solubility of the Tau and Fyn fusions was sufficient for a functional interaction leading to re-assembly or re-constitution of the GFP.

It will be understood that the detectable signal may include enzymatic activity, bioluminescence, chemiluminescence, fluorescence or absorbance, in which case fragments of the relevant signalling moiety, or component parts that give rise to signalling can be provided on Tau and/or Fyn.

Preferably, the Tau domain and Fyn domain are each fused to a portion of a GFP molecule such that association of the Tau and Fyn domain reconstitute or reassemble the GFP allowing for fluorescence to be generated. Preferably, the Tau domain is joined, operably linked, or fused to N-GFP and the Fyn domain is joined to C-GFP, or vice versa.

In one embodiment, determining whether a detectable signal is generated includes determining the quality or quantity of the detectable signal.

Typically the Tau protein and Fyn protein are provided in a cell by expression of a Tau or Fyn-encoding construct in a cell. Any eukaryotic cell line can be used for this purpose, depending on the type of construct used for expression of Tau and Fyn protein. Typically the cell line is negative for Tau and Fyn.

Where the Tau and Fyn protein are provided by recombinant means, there is requirement for nucleic acids, constructs and cells containing same. Therefore, in one embodiment′, there is provided a nucleic acid that includes, consists essentially of or consists of a nucleotide sequence that encodes a Fyn domain as described herein.

Preferably the nucleotide sequence is at least 60, 70, 80, 90, 91, 92, 93, 94, 95, 96, 97, 98, 99 or 100% identical to the sequence shown in SEQ ID NO: 3 or a functionally active fragment or variant thereof.

In a further embodiment, there is provided a nucleic acid that includes, consists essentially of or consists of a nucleotide sequence that encodes a Tau domain as described herein. Preferably the nucleotide sequence is at least 60, 70, 80, 90, 91, 92, 93, 94, 95, 96, 97, 98, 99 or 100% identical to the sequence shown in SEQ ID NO: 4 or a functionally active fragment or variant thereof.

In another embodiment, there is provided a genetic construct including a nucleic acid as described herein. The genetic construct allows expression of the Tau domain and/or Fyn domain in a cell. Preferably the Tau domain and Fyn domain is expressed in a compartment adjacent a post synaptic or dendritic membrane.

In a further embodiment, there is provided a cell including a genetic construct or nucleic acid as described herein. Preferably the cell is a neuronal cell. The neuronal cell may be an immortalized or transformed neuronal cell or a primary neuronal cell. A primary neuronal cell is a neuronal cell that can differentiate into other types of neuronal cells, such as glial cells. A neuronal cell includes unipolar, pseudounipolar, bipolar and multipolar neurons, Basket cells, Betz cells, medium spiny neurons, Purkinje cells, pyramidal cells, Renshaw cells and granule cells. The cell may also be other cells found in the brain including glial cells, such as microglia, astrocytes, oligodendrocytes. The cell may be macrophages have the capacity to or have entered the brain. The cells may be other cells of the central nervous system. The cell may be a cell line including neuroblastoma cells of human or non-human origin or any nerve cell lines available from the ATCC (American Type Culture Collection). An example of a neuroblastoma cell line is SH-SY5Y.

In another embodiment the Tau domain and Fyn domain are recombinant or synthetic and incubated under conditions that allow non-covalent association. Typically the conditions that allow non-covalent association are physiological conditions, or the like, as described further herein.

The third step of the method involves contacting the Tau protein and the Fyn protein with the compound in conditions for permitting the compound to bind to either or both of the Tau protein and Fyn protein, thereby inhibiting the binding of the Tau protein to the Fyn protein when the compound is bound to either or both of Tau protein and Fyn protein. Where a cell is used to provide Tau and Fyn, the Tau and Fyn may be bound to one another before they are contacted with the compound, in accordance with the third step of the method.

The final step in the method involves determining whether a detectable signal is formed from binding of Tau protein to Fyn protein. The means required for this determination are based on the type of signal to be generated. In an embodiment of the invention when Tau and Fyn proteins are fused to a component of GFP such that the binding of Tau protein to Fyn protein permits reassembly or reconstitution of GFP, the detection method may be fluorescent microscopy.

According to the method, where there is an absence of a detectable signal, this indicates that the compound inhibits the binding of the Tau protein to the Fyn protein and therefore indicates a likelihood that the compound reduces excitotoxic signalling in a cell.

Preferably, control assays are run concurrently with a method of the invention to minimise the identification of false positives. For example, the test compound is incubated in the absence of the Tau domain and Fyn domain and instead only in the presence of the two components which act as a split reporter system that when associated generate the detectable signal. This control assay allows for the determination of whether a test compound disrupts the interaction of the components of the split reporter system directly by either binding to one or both of the components of the split reporter system rather than by disrupting the non-covalent association between the Tau domain and Fyn domain.

An assay of the invention can be conducted in high throughput. The assay may be conducted in a cell or in a cell-free environment, such as in a cell lysate or in an in vitro condition. For example, the assay may be performed by using 96 or 384 well plates and the inhibitor may be from a chemical library. Typically, the chemical library is one which has been designed to inhibit protein-protein interactions and/or includes compounds that are known to inhibit protein-protein interactions or are already approved for clinical use in humans or animals.

In one embodiment of the invention there is provided a method for determining whether a compound reduces excitotoxic signalling including the steps of:
providing a compound for which a capacity to reduce excitotoxic signalling is to be determined;
providing a Tau protein and a Fyn protein, wherein the Tau protein includes a domain for binding the Fyn protein, and wherein the Tau and/or Fyn proteins are adapted to form a detectable signal when the Tau protein is bound to the Fyn protein;
contacting the Tau protein or the Fyn protein with the compound in conditions for permitting the compound to bind to either or both of the Tau protein and Fyn protein, thereby inhibiting the binding of the Tau protein to the Fyn protein when the compound is bound to either or both of Tau protein and Fyn protein;
determining whether a detectable signal is formed from binding of Tau protein to Fyn protein;
   wherein an absence of a detectable signal indicates that the compound inhibits the binding of the Tau protein to the Fyn protein,
   thereby determining whether the compound reduces excitotoxic signalling.

The above described methods relate to the identification of compounds likely to inhibit excitotoxicity. The following methods describe the identification of inhibitors of the Tau/Fyn interaction that are likely to selectively inhibit excitotoxicity, while leaving normal constitutive signalling through the NMDA receptors substantially unaffected.

In more detail, many pathologies are caused by, or associated with, aberrant up-regulation or amplification of an endogenous signalling pathway that in a normal physiological state is critical for cell function. While a reduction in the signal transmitted via these up-regulated or amplified pathways is desirable for therapeutic treatment, it is often undesirable to completely ablate the signalling pathway or reduce it to a level below which is required for normal cell function. As described herein, the inventors have developed an assay which unexpectedly provides a means for identifying compounds that reduce pathological signal transduction while still allowing a level of signalling required for normal cell function.

The assay is based on the concept that compounds that permanently disrupt the interaction between Tau and Fyn, for example by irreversibly binding to either or both of Tau or Fyn to prevent their association, would inhibit the excitotoxic signal but also ablate the signalling to an undesirable level. In addition, other pathways where the interaction of Tau and Fyn is required would also be ablated. Many of these pathways may be critical for normal cell function and complete inhibition of signal transmission would be an undesirable characteristic of any inhibitor of the Tau and Fyn interaction. Hence, it is desirable to determine whether an inhibitor of the Tau/Fyn interaction reversibly binds to either or both of Tau and Fyn. Inhibitors that reversibly bind to either or both of Tau and Fyn reduce, inhibit or ameliorate the amplified signal which gives rise to excitotoxicity but allow signalling, that is dependent on Tau and Fyn interacting, and critical for normal cell function to occur.

Therefore, in accordance with the invention there is provided a method for determining whether a compound that inhibits binding of Tau to Fyn is likely to selectively reduce excitotoxic signalling comprising the steps of:
providing an inhibitor in the form of a compound that binds to either or both of Tau and Fyn, thereby inhibiting the binding of Tau to Fyn when the compound is bound to either or both of Tau and Fyn;
providing a Tau protein and a Fyn protein, wherein the Tau protein includes a domain for binding to the Fyn protein, and wherein the Tau and/or Fyn proteins are adapted to form a detectable signal when the Tau protein is bound to the Fyn protein;
utilizing the inhibitor to inhibit binding of the Tau protein to the Fyn protein, thereby inhibiting generation of a detectable signal that is formed from binding of the Tau protein to the Fyn protein;
providing conditions for permitting formation of the detectable signal; and
determining whether a detectable signal is formed,
   wherein formation of the detectable signal determines that the compound is likely to selectively reduce excitotoxic cell signalling.

Formation of, or recovery of the detectable signal after the Tau and Fyn domains are contacted with the inhibitor determines that the inhibitor does not permanently disrupt the association of the Tau domain and Fyn domain. Further, recovery of the detectable signal indicates that the inhibitor reduces pathophysiological excitotoxic cell signalling to a non-cytotoxic level. If the method is conducted intracellularly then recovery of the signal also indicates that the inhibitor is not cytotoxic in a Tau and Fyn independent manner. Recovery of the detectable signal includes restoration or regeneration of the detectable signal to a level or degree that is similar or the same as the detectable signal that would occur if a Tau domain of the invention and Fyn domain of the invention are allowed to associate in the absence of an inhibitor.

Providing conditions for the formation of, or recovery of the detectable signal after the Tau and Fyn domains are contacted with the inhibitor is intended to determine whether the inhibitor can dissociate from Tau or Fyn or both over time thereby allowing Tau and Fyn to interact. The conditions which allow this to occur are typically physiological conditions, or the like, thereby allowing determination as to whether the inhibitor interferes with only the pathophysiological excitiotoxic signalling mediated by Tau and Fyn or also inhibits the normal physiological signalling via Tau and Fyn. Physiological conditions may include one or more of the following, a temperature range of 20 to 40 degrees Celsius, preferably about 37 degrees Celsius, atmospheric pressure of 1, pH of 6 to 8, preferably a pH of 7.0 to 7.5, glucose concentration of 1 to 20 mM, atmospheric oxygen concentration and about 10% carbon dioxide concentration. These conditions particularly apply when the Tau and Fyn protein are derived from a mammalian sequence and/or the assay is performed in a mammalian system, e.g. a mammalian cell, or lysate from a mammalian cell. These conditions also apply if the assay is conducted in vitro, non-intracellular, cell free environment.

In further embodiments there is provided a kit for use in an assay of the invention, the kit including (a) a Tau domain and (b) a Fyn domain. Preferably, the kit includes a nucleic acid as described herein encoding a Tau domain and a nucleic acid as described herein encoding a Fyn domain. Preferably, the kit includes instructions directing an individual to perform a method of the invention.

In one embodiment, the present invention provides a kit as described above when used in a method of the invention.

It will be understood that these examples are intended to demonstrate these and other aspects of the invention and although the examples describe certain embodiments of the invention, it will be understood that the examples do not limit these embodiments to these things. Various changes can be made and equivalents can be substituted and modifications made without departing from the aspects and/or principles of the invention mentioned above. All such changes, equivalents and modifications are intended to be within the scope of the claims set forth herein.

### EXAMPLES

### Example 1

This Example describes the generation of expression vectors containing N-GFP and C-GFP fused to a portion of either Fyn or Tau and the transfection of cells with those vectors.

In preparing expression vectors encoding N-GFP and C-GFP fused to a Fyn or Tau protein the inventors had to take into consideration a number of factor including, the amino acid sequence and length of the Fyn and Tau protein, whether to fuse the GFP component to either the N or C-terminus of the Fyn or Tau protein, whether to fuse the N- or C-GFP to Fyn or to Tau, and the presence and length of a flexible linker between the GFP component and Fyn or Tau protein.

N-GFP and C-GFP vectors were generated according to Ghosh et al., J Am Chem Soc.

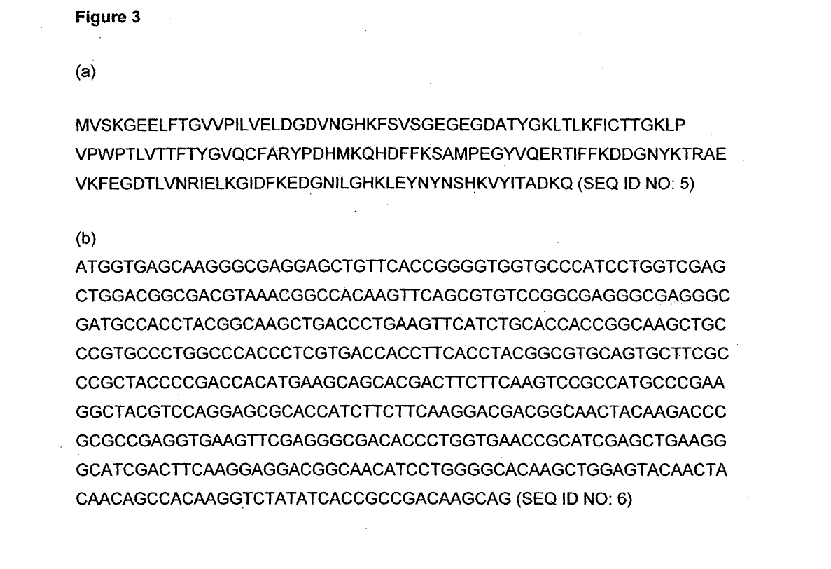

Generation of N-GFP: cDNA encoding aa 1-158 of GFP (FIG. 3 (a) amino acid sequence and FIG. 3(b) nucleotide sequence) were cloned into pLVX (Clontech) by PCR using EcoRI (G′AATCC) and XbaI (T′CTAGA). A Kozac sequence ACC was also present. A nucleotide sequence encoding a 4 amino acid linker (GGCGGCTCCGGC) was added to the 3′ end of the nucleotide sequence encoding the N-GFP fragment to later link the interaction domain of interest. Primers used in the construction of the N-GFP vector were N-GFP-F, GGGAATTCACCATGGTGAGCAAGG GCGAGGAG (SEQ ID NO: 9), and N-GFP-R, GGTCTAGAGCCGGAGCCGCCCTGCTTGTCGG CGGTGATATAG (SEQ ID NO: 10).

The nucleotide sequence of the N-GFP construct, showing the restriction sites, N-GFP sequence and linker, and the encoded amino acid sequence are shown below:

(SEQ ID NO: 11)
GAATTCACCATGGTGAGCAAGGGCGAGGAGCTGTTCACCGGGGTGGTGC
CCATCCTGGTCGAGCTGGACGGCGACGTAAACGGCCACAAGTTCAGCGT
GTCCGGCGAGGGCGAGGGCGATGCCACCTACGGCAAGCTGACCCTGAAG
TTCATCTGCACCACCGGCAAGCTGCCCGTGCCCTGGCCCACCCTCGTGA
CCACCTTCACCTACGGCGTGCAGTGCTTCGCCCGCTACCCCGACCACAT
GAAGCAGCACGACTTCTTCAAGTCCGCCATGCCCGAAGGCTACGTCCAG
GAGCGCACCATCTTCTTCAAGGACGACGGCAACTACAAGACCCGCGCCG
AGGTGAAGTTCGAGGGCGACACCCTGGTGAACCGCATCGAGCTGAAGGG
CATCGACTTCAAGGAGGACGGCAACATCCTGGGGCACAAGCTGGAGTAC
AACTACAACAGCCACAAGGTCTATATCACCGCCGACAAGCAGGGCGGCT
CCGGCTCTAGA
(SEQ ID NO: 12)
MVSKGEELFTGVVPILVELDGDVNGHKFSVSGEGEGDATYGKLTLKFIC
TTGKLPVPWPTLVTTFTYGVQCFARYPDHMKQHDFFKSAMPEGYVQERT
IFFKDDGNYKTRAEVKFEGDTLVNRIELKGIDFKEDGNILGHKLEYNYN
SHKVYITADKQGGSGSR

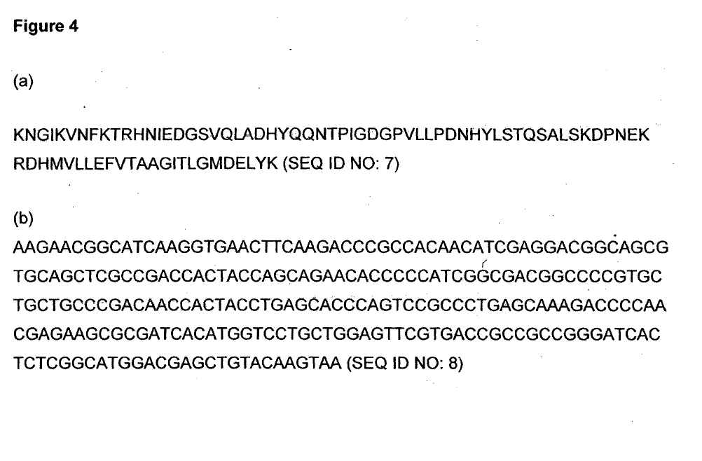

Generation of N-terminal C-GFP: cDNA encoding aa 159-239 of GFP (FIG. 4(a) amino acid sequence and FIG. 4(b) nucleotide sequence) were cloned into pLVX (Clontech) by PCR using EcoRI (G′AATCC) and XbaI (T′CTAGA). A Kozac sequence ACC was also present. A nucleotide sequence encoding a 4 amino acid linker (GGCGGCTCCGGC) was added to the 3′ end of the nucleotide sequence encoding the C-GFP fragment to later link the interaction domain of interest. Primers used in the construction of the N-terminal C-GFP vector were nC-GFP-F, GGGAATTCACCATGAAGAACGGCATCAAGGTGAAC (SEQ ID NO: 13), and nC-GFP-R, GGTCTAGAGCCGGAGCCGCCCTTGTACAGCTCGTCCATGCCG (SEQ ID NO: 14).

The nucleotide sequence of the N-terminal C-GFP construct, showing the restriction sites, C-GFP sequence and linker, and the encoded amino acid sequence are shown below:

(SEQ ID NO: 15)
GAATTCACCATGAAGAACGGCATCAAGGTGAACTTCAAGACCCGCCACA
ACATCGAGGACGGCAGCGTGCAGCTCGCCGACCACTACCAGCAGAACAC
CCCCATCGGCGACGGCCCCGTGCTGCTGCCCGACAACCACTACCTGAGC
ACCCAGTCCGCCCTGAGCAAAGACCCCAACGAGAAGCGCGATCACATGG
TCCTGCTGGAGTTCGTGACCGCCGCCGGGATCACTCTCGGCATGGACGA
GCTGTACAAGGGCGGCTCCGGCTCTAGA
(SEQ ID NO: 16)
MKNGIKVNFKTRHNIEDGSVQLADHYQQNTPIGDGPVLLPDNHYLSTQS
ALSKDPNEKRDHMVLLEFVTAAGITLGMDELYKGGSGSR

Generation of C-terminal C-GFP: cDNA encoding aa 159-239 of GFP (FIG. 4(a) amino acid sequence and FIG. 4(b) nucleotide sequence) were cloned into pLVX (Clontech) by PCR using XbaI (T′CTAGA) and BamHI (G′GATCC). A nucleotide sequence encoding a 4 amino acid linker (GGCGGCTCCGGC) was added to the 5′ end of the nucleotide sequence encoding the C-GFP fragment to later link the interaction domain of interest. Primers used in the construction of the C-terminal C-GFP vector were cC-GFP-F, GGTCTAGAGGCGGCTCCGGCAAGAACGGCATCAAGGTGAACTTC (SEQ ID NO: 17), and cC-GFP-R, GGGGATCCTTACTTGTACAGCTCGTCCATGC (SEQ ID NO: 18).

The nucleotide sequence of the C-terminal C-GFP construct, showing restrictions sites, C-GFP and linker, and the encoded amino acid sequence are shown below:

(SEQ ID NO: 19)
TCTAGAGGCGGCTCCGGCAAGAACGGCATCAAGGTGAACTTCAAGACCC
GCCACAACATCGAGGACGGCAGCGTGCAGCTCGCCGACCACTACCAGCA
GAACACCCCCATCGGCGACGGCCCCGTGCTGCTGCCCGACAACCACTAC
CTGAGCACCCAGTCCGCCCTGAGCAAAGACCCCAACGAGAAGCGCGATC
ACATGGTCCTGCTGGAGTTCGTGACCGCCGCCGGGATCACTCTCGGCAT
GGACGAGCTGTACAAGTAAGGATCC
(SEQ ID NO: 20)
SRGGSGKNGIKVNFKTRHNIEDGSVQLADHYQQNTPIGDGPVLLPDNHY
LSTQSALSKDPNEK RDHMVLLEFVTAAGITLGMDELYK

A nucleic acid encoding amino acids 83-145 of human Fyn was cloned into the N-GFP-pLVX vector. The nucleic acid encodes the SH3 domain of Fyn:

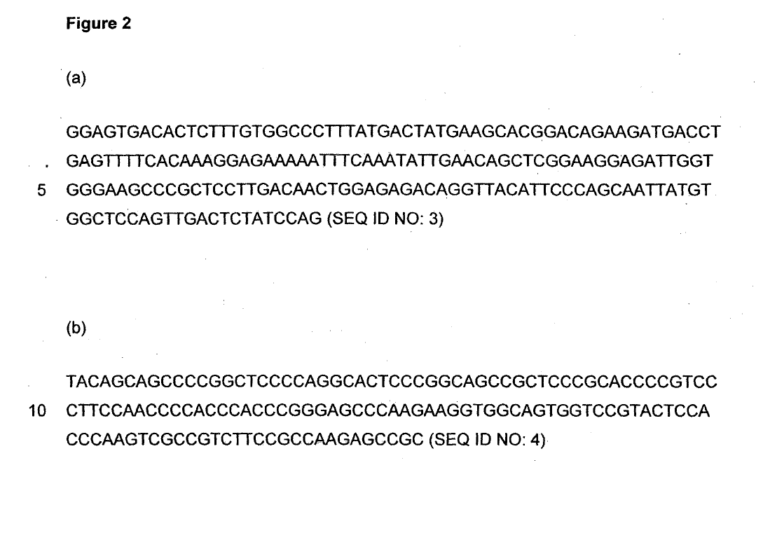

(SEQ ID NO: 3, FIG. 2a)
GGAGTGACACTCTTTGTGGCCCTTTATGACTATGAAGCACGGACAGAAG
ATGACCTGAGTTTTCACAAAGGAGAAAAATTTCAAATATTGAACAGCTC
GGAAGGAGATTGGTGGGAAGCCCGCTCCTTGACAACTGGAGAGACAGGT
TACATTCCCAGCAATTATGTGGCTCCAGTTGACTCTATCCAG.

A nucleic acid encoding amino acids 197-242 of human Tau (2N4R) was cloned into both the N-terminal and C-terminal C-GFP-pLVX vectors. The nucleic acid encodes for the PXXP domain of Tau

(SEQ ID NO: 4, FIG. 2b).
TACAGCAGCCCCGGCTCCCCAGGCACTCCCGGCAGCCGCTCCCGCACCC
CGTCCCTTCCAACCCCACCCACCCGGGAGCCCAAGAAGGTGGCAGTGGT
CCGTACTCCACCCAAGTCGCCGTCTTCCGCCAAGAGCCGC.

Lentiviruses containing either combination of constructs, i.e. N-terminal C-GFP fused to Tau and N-GFP fused to Fyn, or C-terminal C-GFP fused to Tau and N-GFP fused to Fyn, were then generated using standard protocols (e.g. Krupka et al., Plasmid, 2010, 63(3):155-60) and stably expressed in COS-7 cells.

The COS-7 cells transfected with constructs expressing C-terminal C-GFP fused to Tau and N-GFP fused to Fyn did not result in green fluorescing cells.

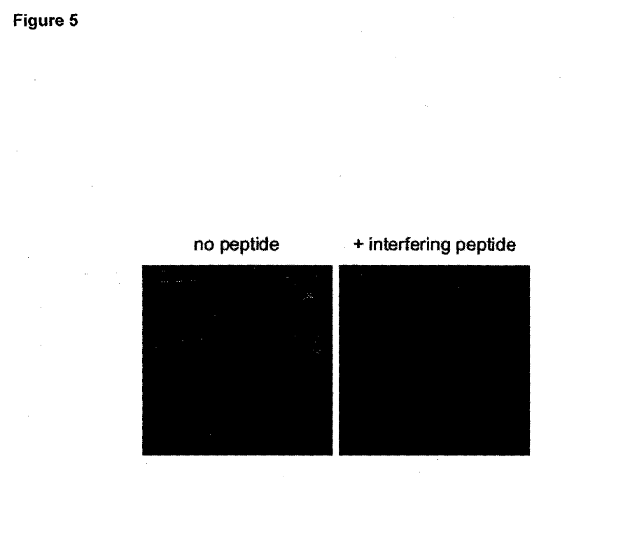

However, transfection of constructs encoding N-terminal C-GFP fused to Tau and N-GFP fused to Fyn did result in green fluorescing cells (FIG. 5). Control cells expressed either the Fyn or the Tau construct and showed no detectable fluorescence. Cells expressing N- and C-GFP together, but no linked interaction domain also showed no fluorescence.

The inability of the combination of C-terminal C-GFP fused to Tau and N-GFP fused to Fyn to generate a fluorescent signal when simultaneously expressed in a cell highlights the difficulty in predicting the likelihood of what Tau and Fyn GFP fusion would work.

### Example 2

This Example describes the proof-of-concept that the assay can be used to identify compounds that reduce the interaction of Fyn with Tau.

Cells generated by the methods described in Example 1 were grown till confluent in 96-well plates and then the fluorescence was measured in a 96-well plate reader (Omega, BMG) or by via fluorescence microscope. The cells were then incubated with 200 nM of the Tat-7PXXP peptide for 1 hour at 37 degree Celsius. The fluorescence was again measured in a 96-well plate reader (Omega, BMG) or via a fluorescence microscope.

The fluorescence of the transfected cells was negligible in the presence of the Tat-7PXXP peptide (FIG. 5). The Tat-7PXXP peptide has the Tat sequence, for cell permeability, linked to Tyr Gly Arg Lys Lys Arg Arg Gln Arg Arg Arg Thr Pro Pro Lys Ser Pro Ser Ser (SEQ ID NO: 21).

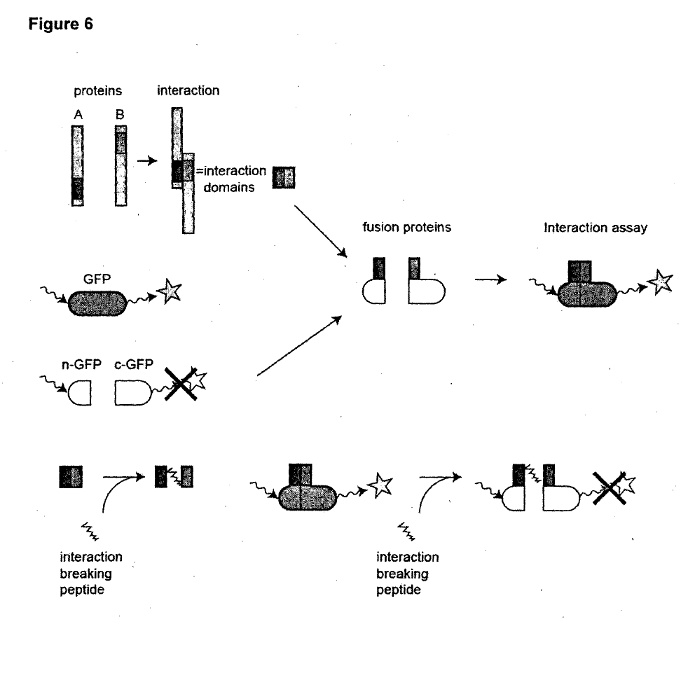

A general schematic of the assay described in the Examples is shown in FIG. 6.

All publications and patents cited in this specification are incorporated by reference as if each individual publication or patent were specifically and individually indicated to be incorporated by reference. Further, any polypeptide sequence, polynucleotide sequences or annotation thereof, are incorporated by reference herein. The citation of any publication is for its disclosure prior to the filing date and should not be construed as an admission that the present invention is not entitled to antedate such publication by virtue of prior invention.

The claims are as follows:

1. A method for determining whether a compound reduces excitotoxic signaling in a cell comprising the steps of:
(a) providing a compound for which a capacity to reduce excitotoxic signaling in a cell is to be determined;
(b) providing a Tau protein and a Fyn protein, wherein the Tau protein includes a domain for binding the Fyn protein, and wherein the Tau and/or Fyn proteins are adapted to form a detectable signal when the Tau protein is bound to the Fyn protein;
(c) contacting the Tau protein and the Fyn protein with the compound in conditions for permitting the compound to bind to either or both of the Tau protein and Fyn protein, thereby inhibiting the binding of the Tau protein to the Fyn protein when the compound is bound to either or both of Tau protein and Fyn protein;
(d) determining whether a detectable signal is formed from binding of Tau protein to Fyn protein;
wherein an absence of a detectable signal indicates that the compound inhibits the binding of the Tau protein to the Fyn protein,
thereby determining whether the compound reduces excitotoxic signaling in a cell.

2. A method for determining whether a compound that inhibits binding of Tau to Fyn is likely to selectively reduce excitotoxic cell signaling comprising the steps of:
(a) providing an inhibitor in the form of a compound that binds to either or both of Tau and Fyn, thereby inhibiting the binding of Tau to Fyn when the compound is bound to either or both of Tau and Fyn;
(b) providing a Tau protein and a Fyn protein, wherein the Tau protein includes a domain for binding to the Fyn protein, and wherein the Tau and/or Fyn proteins are adapted to form a detectable signal when the Tau protein is bound to the Fyn protein;
(c) utilizing the inhibitor to inhibit binding of the Tau protein to the Fyn protein, thereby inhibiting generation of a detectable signal that is formed from binding of the Tau protein to the Fyn protein;
(d) providing conditions for permitting formation of the detectable signal; and
(e) determining whether the a detectable signal is formed,
wherein formation of the detectable signal determines that the compound is likely to selectively reduce excitotoxic cell signaling.

3. A method according to claim 1, wherein the Tau protein consists of the seventh MOO motif of the amino terminal projection domain.

4. A method according to claim 1, wherein the Tau protein consists of the amino terminal projection domain.

5. A method according to claim 1, wherein the Tau protein includes an amino acid sequence that is 60, 70, 80, 90, 91, 92, 93, 94, 95, 96, 97, 98, 99 or 100% identical to the sequence shown in SEQ ID NO: 2.

6. A method according to claim 1, wherein the Tau protein consists of an amino acid sequence that is 60, 70, 80, 90, 91, 92, 93, 94, 95, 96, 97, 98, 99 or 100% identical to the sequence shown in SEQ ID NO: 2.

7. A method according to claim 1, wherein Fyn consists of the SH3 domain.

8. A method according to claim 1, wherein the Fyn protein includes an amino acid sequence that is 60, 70, 80, 90, 91, 92, 93, 94, 95, 96, 97, 98, 99 or 100% identical to the sequence shown in SEQ ID NO: 1.

9. A method according to claim 1, wherein the Fyn protein consists of an amino acid sequence that is 60, 70, 80, 90, 91, 92, 93, 94, 95, 96, 97, 98, 99 or 100% identical to the sequence shown in SEQ ID NO: 1.

10. A method according to claim 1, wherein the detectable signal is enzymatic activity, bioluminescence, chemiluminescence, fluorescence or absorbance.

11. A method according to claim 10, wherein the detectable signal is fluorescence generated from GFP.

12. A method according to claim 11, wherein the GFP is formed from re-assembly or re-constitution by an N terminal portion of GFP fused to the Fyn protein and a C terminal portion of GFP fused to the Tau protein.

13. A nucleic acid encoding:
(a) a Tau protein that is adapted to form a detectable signal when the Tau protein is bound to a Fyn protein; or
(b) a Fyn protein that is adapted to form a detectable signal when the Fyn protein is bound to a Tau protein.

14. A nucleic acid according to claim 13, wherein the Tau protein is encoded by SEQ ID NO: 4.

15. (canceled)

16. A nucleic acid according to claim 13, wherein the Fyn protein is encoded by SEQ ID NO: 3.

17. A vector or construct including a nucleic acid according to claim 13.

18. (canceled)
